(19) United States
(12) Patent Application Publication
(10) Pub. No.: US 2025/0372257 A1
(43) Pub. Date: Dec. 4, 2025

(54) FOUR-COMPARTMENT DIFFUSION MODEL OF INSULIN IN HUMANS

(71) Applicant: United States Government As Represented By The Department of Veterans Affairs, Washington, DC (US)

(72) Inventors: Richard I. Dorin, Albuquerque, NM (US); Clifford R. Qualls, Albuquerque, NM (US)

(21) Appl. No.: 19/225,745

(22) Filed: Jun. 2, 2025

Related U.S. Application Data
(60) Provisional application No. 63/654,616, filed on May 31, 2024.

(51) Int. Cl. G16H 50/20; A61B 5/00

(52) U.S. Cl. CPC G16H 50/20; A61B 5/4866

(57) ABSTRACT

Methods, systems, and apparatuses for modeling and determining of an insulin elimination rate in individuals are described. One or more physiological measurements associated with an individual may be determined and applied to a multi-compartment model. One or more physiological parameters, including an insulin elimination rate, may be determined based on applying the one or more physiological measurements to the multi-compartment model.

### CROSS REFERENCE TO RELATED PATENT APPLICATION

This application claims priority to U.S. Provisional Application No. 63/654,616, filed May 31, 2024, which is herein incorporated by reference in its entirety.

### BACKGROUND

Modeling insulin elimination typically involves the use of compartmental models, often used to understand how insulin is absorbed, distributed, and eliminated from the body. These models often involve one-, or two-, compartment models, with varying complexities depending on the desired level of detail and the specific physiologic process being studied. Metabolic elimination of insulin (I) in vivo includes pathways mediated by high-affinity and potentially saturable insulin receptor binding. Endogenous insulin secreted into the portal vein is subject to first pass elimination. The rate of insulin flux between vascular (Vp) and interstitial (Vi) compartments is often modeled by first order rate constants. These models consider absorption processes and how insulin is subsequently cleared by the liver and kidneys. These models also incorporate insulin sensitivity, which refers to how effectively insulin can lower blood glucose levels in target tissues such as the liver, muscle, and adipose tissue. Model parameters, such as absorption rate, elimination rate, and insulin sensitivity, are estimated using experimental data, such as plasma insulin and glucose levels measured after insulin administration or after a glucose challenge. These compartment models divide the body into compartments (e.g., plasma tissues) and simulate the movement of insulin between the compartments, as well as its elimination. One-compartment models assume insulin is distributed uniformly throughout the body and eliminated at a single rate. Other compartment models account for the presence of different tissues and organs that have varying insulin uptake and clearance rates. However, there is uncertainty about which of the models in current use, if any, are sufficiently accurate for clinical application. This disclosure aims to tackle these issues by introducing novel approaches to calculating insulin elimination rates in a human body.

### SUMMARY

It is to be understood that both the following general description and the following detailed description are exemplary and explanatory only and are not restrictive.

Methods, apparatuses, and systems for determining an insulin elimination rate in an individual are described. One or more physiological measurements associated with an individual may be determined and applied to a multi-compartment model. One or more physiological parameters, including an insulin elimination rate, may be determined based on applying the one or more physiological measurements to the multi-compartment model.

In an embodiment, disclosed are methods comprising determining, by a computing device, one or more physiological measurements associated with an individual, applying the one or more physiological measurements to a multi-compartment model, wherein the multi-compartment model represents a bidirectional flux by diffusion of a hormone between a vascular compartment, an interstitial compartment, a hepatic compartment, and a renal compartment of the individual, determining, based on the application of the one or more physiological measurements to the multi-compartment model, one or more physiological parameters associated with the individual, and determining, based on the one or more physiological parameters, one or more medical conditions associated with the individual.

In an embodiment, disclosed are methods comprising determining, by a computing device, one or more physiological measurements associated with an individual, applying the one or more physiological measurements to a multi-compartment model, wherein the multi-compartment model represents a bidirectional flux by diffusion of a hormone between a vascular compartment, an interstitial compartment, a hepatic compartment, and a renal compartment of the individual, determining, based on the application of the one or more physiological measurements to the multi-compartment model, one or more physiological parameters associated with the individual, and administering, based on the one or more physiological parameters, a treatment associated with one or more medical conditions associated with the individual.

Additional advantages will be set forth in part in the description which follows or may be learned by practice. The advantages will be realized and attained by means of the elements and combinations particularly pointed out in the appended claims.

### BRIEF DESCRIPTION OF THE DRAWINGS

The accompanying drawings, which are incorporated in and constitute a part of the present description serve to explain the principles of the methods and systems described herein:

FIG. 1 shows an example system for determining an insulin elimination rate;

FIG. 2 shows an example of blood plasma flow between different compartments;

FIG. 3 shows an example graphical solution;

FIG. 4 shows an example graphical solution;

FIG. 5 shows an example system environment;

FIG. 6 shows a flowchart of an example method;

FIG. 7 shows a flowchart of an example method; and

FIG. 8 shows a block diagram of an example system and computing device for determining an insulin elimination rate.

### DETAILED DESCRIPTION

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another configuration includes from the one particular value and/or to the other particular value. Similarly, when values are expressed as approximations, by use of the antecedent “about,” it will be understood that the particular value forms another configuration. It will be further understood that the endpoints of each of the ranges are significant both in relation to the other endpoint, and independently of the other endpoint.

“Optional” or “optionally” means that the subsequently described event or circumstance may or may not occur, and that the description includes cases where said event or circumstance occurs and cases where it does not.

As used herein the terms “individual,” “patient,” “subject,” “user,” or “person” may indicate a person associated with the determination of one or more physiological parameters, such as an interstitial insulin elimination rate, a hepatic insulin elimination rate, a renal insulin elimination rate, or a permeability constant related to insulin diffusion in the human body.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal configuration. “Such as” is not used in a restrictive sense, but for explanatory purposes.

It is understood that when combinations, subsets, interactions, groups, etc. of components are described that, while specific reference of each various individual and collective combinations and permutations of these may not be explicitly described, each is specifically contemplated and described herein. This applies to all parts of this application including, but not limited to, steps in described methods. Thus, if there are a variety of additional steps that may be performed it is understood that each of these additional steps may be performed with any specific configuration or combination of configurations of the described methods.

As will be appreciated by one skilled in the art, hardware, software, or a combination of software and hardware may be implemented. Furthermore, the methods and systems may take the form of a computer program product on a computer-readable storage medium (e.g., non-transitory) having processor-executable instructions (e.g., computer software) embodied in the storage medium. More particularly, the present methods and systems may take the form of web-implemented computer software. Any suitable computer-readable storage medium may be utilized including hard disks, CD-ROMs, optical storage devices, magnetic storage devices, memresistors, Non-Volatile Random Access Memory (NVRAM), flash memory, or a combination thereof.

Throughout this application reference is made to block diagrams and flowcharts. It will be understood that each block of the block diagrams and flowcharts, and combinations of blocks in the block diagrams and flowcharts, respectively, may be implemented by processor-executable instructions. These processor-executable instructions may be loaded onto a general purpose computer, special purpose computer, or other programmable data processing apparatus to produce a machine, such that the processor-executable instructions which execute on the computer or other programmable data processing apparatus create a device for implementing the functions specified in the flowchart block or blocks. In addition, some of these functions may be carried out using complex programmable logic devices (CPLDs) or other programmable logic devices.

The processor-executable instructions may also be stored in a computer-readable memory that may direct a computer or other programmable data processing apparatus to function in a particular manner, such that the processor-executable instructions stored in the computer-readable memory produce an article of manufacture including processor-executable instructions for implementing the function specified in the flowchart block or blocks. The processor-executable instructions may also be loaded onto a computer or other programmable data processing apparatus to cause a series of operational steps to be performed on the computer or other programmable apparatus to produce a computer-implemented process such that the processor-executable instructions that execute on the computer or other programmable apparatus provide steps for implementing the functions specified in the flowchart block or blocks. In addition, some of these functions may be carried out using logic devices which do not operate by sequential operations of programmed steps.

Blocks of the block diagrams and flowcharts support combinations of devices for performing the specified functions, combinations of steps for performing the specified functions and program instruction means for performing the specified functions. It will also be understood that each block of the block diagrams and flowcharts, and combinations of blocks in the block diagrams and flowcharts, may be implemented by special purpose hardware-based computer systems that perform the specified functions or steps, or combinations of special purpose hardware and computer instructions and logic circuitry.

Methods and systems are described for determining one or more physiological parameters of a human subject, such as an interstitial insulin elimination rate, a hepatic insulin elimination rate, a renal insulin elimination rate, and a permeability constant related to insulin diffusion. The physiological parameters are important for predicting one or more physiological attributes associated with an individual, such as the concentration of insulin in one or more of a vascular volume, an interstitial volume, a hepatic volume, and/or a renal volume. For example, one or more physiological measurements associated with an individual may be determined. The one or more physiological measurements may comprise one or more of plasma insulin concentrations, a plasma volume, a vascular volume, an interstitial volume, a hepatic volume, or a renal volume. For example, the one or more physiological measurements may be applied to a multi-compartment. The multi-compartment model may represent a bidirectional flux by diffusion of a hormone between a vascular compartment, an interstitial compartment, a hepatic compartment, and a renal compartment of the individual. The hormone may comprise one or more of insulin, C-peptide, glucagon, amylin, somatostatin, insulin-like growth factors (IGFs), or incretins. The multi-compartment model may comprise four non-linear differential equations. The four non-linear differential equations may represent time-varying concentrations of insulin in a vascular plasma volume, in an interstitial volume, in a hepatic volume, and in a renal volume. The one or more physiological parameters may be determined based on the application of the one or more physiological measurements to the multi-compartment model. One or more medical conditions, such as diabetes, insulin resistance, hypoglycemia, insulinoma, Cushing's syndrome, acromegaly, hypothyroidism, hypertension, dyslipidemia, hyperuricemia, or endothelial dysfunction, associated with the individual may be determined based on the one or more physiological parameters. In addition, a treatment may be administered or adjusted, such as an administration or adjustment of the hormone or an administration or adjustment of a hormone replacement therapy, based on the one or more medical conditions.

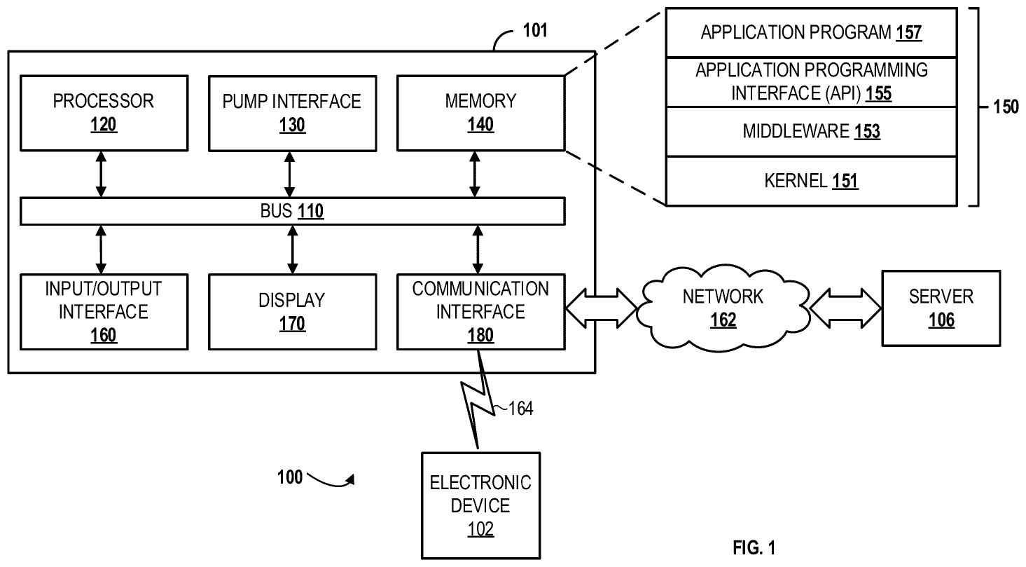

FIG. 1 shows an example system 100 for determining one or more physiological parameters of a human subject (e.g., patient, individual, etc.), such as an interstitial insulin elimination rate, a hepatic insulin elimination rate, a renal insulin elimination rate, and a permeability constant related to insulin diffusion. The system 100 may be configured to process one or more physiological measurements associated with an individual and apply the physiological measurements to a multi-compartment model to determine the one or more physiological parameters associated with the individual. Referring to FIG. 1, the system 100 may comprise a device 101. The device 101 may comprise, for example, a mobile phone, a smart phone, a tablet computer, a laptop, a desktop computer, a smartwatch, a smart glass, an insulin pump device, and the like. As an example, the device 101 may comprise a computing device for controlling an insulin pump. As an example, the device 101 and the insulin pump may be integrated together as a single device or may comprise separate devices. The device 101 may be configured to control the insulin pump (e.g., via a pump interface 130) for delivering insulin to a human subject/individual via tubing connected between the insulin pump and an infusion set affixed/attached to a location of the individual's body. The device 101 may include a bus 110, one or more processors 120, a pump interface 130, a memory 140, an input/output interface 160, a display 170, and a communication interface 180. In an example, the device 101 may omit at least one of the aforementioned constitutional elements or may additionally include other constitutional elements.

The bus 110 may include a circuit for connecting the bus 110, the one or more processors 120, the pump interface 130, the memory 140, the input/output interface 160, the display 170, and/or the communication interface 180 to each other and for delivering communication (e.g., a control message and/or data) between the bus 110, the one or more processors 120, the pump interface 130, the memory 140, the input/output interface 160, the display 170, and/or the communication interface 180.

The one or more processors 120 may include one or more of a Central Processing Unit (CPU), an Application Processor (AP), and a Communication Processor (CP). The one or more processors 120 may control, for example, at least one of the bus 110, the pump interface 130, the memory 140, the input/output interface 160, the display 170, and/or the communication interface 180 and/or may execute an arithmetic operation or data processing for communication. The processing (or controlling) operation of the one or more processors 120 according to various embodiments is described in detail with reference to the following drawings.

The processor-executable instructions executed by the one or more processor 120 may be stored and/or maintained by the memory 140. The memory 140 may include a volatile and/or non-volatile memory. The memory 140 may store, for example, a command or data related to at least one different constitutional element of the electronic device 101. According to various exemplary embodiments, the memory 140 may store a software and/or a program 150. The program 150 may include, for example, a kernel 151, a middleware 153, an Application Programming Interface (API) 155, and/or an application program (or an “application”) 157, or the like, configured for controlling one or more functions of the device 101 and/or an external device. At least one part of the kernel 151, middleware 153, or API 155 may be referred to as an Operating System (OS). The memory 140 may include a computer-readable recording medium having a program recorded therein to perform the method according to various embodiments by the processor 120.

The kernel 151 may control or manage, for example, system resources (e.g., the bus 110, the processor 120, the memory 140, etc.) used to execute an operation or function implemented in other programs (e.g., the middleware 153, the API 155, or the application program 157). Further, the kernel 151 may provide an interface capable of controlling or managing the system resources by accessing individual constitutional elements of the device 101 in the middleware 153, the API 155, or the application program 157.

The middleware 153 may perform, for example, a mediation role so that the API 155 or the application program 157 can communicate with the kernel 151 to exchange data. Further, the middleware 153 may handle one or more task requests received from the application program 157 according to a priority. For example, the middleware 153 may assign a priority of using the system resources (e.g., the bus 110, the processor 120, or the memory 140) of the device 101 to at least one of the application programs 157. For instance, the middleware 153 may process the one or more task requests according to the priority assigned to the at least one of the application programs, and thus may perform scheduling or load balancing on the one or more task requests.

The API 155 may include at least one interface or function (e.g., instruction), for example, for file control, window control, video processing, or character control, as an interface capable of controlling a function provided by the application 157 in the kernel 151 or the middleware 153.

The application program 157 may include logic (e.g., hardware, software, firmware, etc.) that may be implemented to determine one or more physiological parameters of an individual. For example, the device 101 may determine one or more physiological measurements associated with an individual. The one or more physiological measurements may comprise one or more of plasma insulin concentrations, a plasma volume, a vascular volume, an interstitial volume, a hepatic volume, or a renal volume. The application program 157 may cause the device 101 to apply the one or more physiological measurements to a multi-compartment model that represents a bidirectional flux by diffusion of a hormone between a vascular compartment (e.g., fluid compartment within the blood vessels), an interstitial compartment (e.g., space and fluid surrounding cells within tissues), a hepatic compartment (e.g., space within the liver), and a renal compartment (e.g., space within the kidney) of the individual. For example, the multi-compartment model may comprise four non-linear differential equations that represent time-varying concentrations of insulin in a vascular plasma volume, in an interstitial volume, in a hepatic volume, and in a renal volume. The hormone may comprise one or more of insulin (e.g., endogenous and/or exogenous insulin), C-peptide, glucagon, amylin, somatostatin, insulin-like growth factors (IGFs), or incretins. The application program 157 may cause the device 101 to determine one or more physiological parameters associated with the individual based on the application of the one or more physiological measurements to the multi-compartment model. The one or more physiological parameters may comprise one or more of an interstitial insulin elimination rate (αᵢ), a hepatic insulin elimination rate (αₕ), a renal insulin elimination rate (αₖ), or a permeability constant related to insulin diffusion (k). The application program 157 may cause the device 101 to determine one or more medical conditions associated with the individual based on the one or more physiological parameters. The one or more medical conditions associated with the individual may comprise one or more of diabetes (e.g., type 1, 2, and/or 3c diabetes), insulin resistance, hypoglycemia, insulinoma, Cushing's syndrome, acromegaly, hypothyroidism, hypertension, dyslipidemia, hyperuricemia, or endothelial dysfunction. In an example, the multi-compartmental model may be utilized to track/monitor endogenous insulin secretion and exogenous insulin administration in individuals/patients with preserved beta cell function (e.g., in individuals/patients with type 2 diabetes mellitus). In an example, measurements of insulin sensitivity and beta cell function may be utilized to predict a probability of future development of diabetes in an individual/patient. For example, the multi-compartmental model may be utilized to increase the accuracy of obtaining clinically useful measures of insulin sensitivity and insulin secretory capacity over other methods, such as HOMA-IR and HOMA-beta. In an example, the one or more medical conditions may be determined based on one or more physiological attributes associated with the individual. For example, the one or more physiological attributes may comprise a concentration of insulin in one or more of a vascular volume, an interstitial volume, a hepatic volume, or a renal volume. The one or more physiological attributes associated with the individual may be determined based on the one or more physiological parameters. As an example, the one or more medical conditions may be determined based on an appearance and elimination rate of the hormone in the individual. For example, the appearance and elimination rate of the hormone in the individual may be determined based on the one or more physiological parameters.

The application program 157 may cause the device 101 to cause one or more treatments associated with the one or more medical conditions associated with the individual to be administered to the individual based on the one or more physiological parameters. As an example, the one or more treatments may be administered based on the one or more physiological attributes associated with the individual. As an example, the one or more treatments may be administered based on the appearance and elimination rate of the hormone in the individual. The one or more treatments may comprise administering the hormone, or hormone replacement therapy. In an example, the hormone may be administered to the individual when the one or more medical conditions associated with the individual are determined. As an example, the administration of the hormone may be adjusted based on the one or more physiological parameters. For example, an inflow of insulin (e.g., via the insulin pump) to the individual may be reduced or increased based on the one or more physiological parameters.

The input/output interface 160 may be configured as an interface for delivering an instruction or data input from a user or a different external device(s) to the processor 120, the memory 140, the display 170, and the communication interface 180. For example, the input/output interface 160 may receive user input for programming the device 101. For example, the input/output interface 160 may receive user input adjusting one or more settings of the device 101 (e.g., settings of the insulin pump). Further, the input/output interface 160 may output an instruction or data received from the processor 120, the memory 140, the input/output interface 160, the display 170, and/or the communication interface 180 to a different external device (e.g., electronic device 102, server 106, etc.).

The display 170 may include various types of displays, for example, a Liquid Crystal Display (LCD) display, a Light Emitting Diode (LED) display, an Organic Light-Emitting Diode (OLED) display, a MicroElectroMechanical Systems (MEMS) display, or an electronic paper display. The display 160 may display, for example, a variety of contents (e.g., text, image, video, icon, symbol, etc.) to the user. The display 160 may include a touch screen. For example, the display 160 may receive a touch, gesture, proximity, or hovering input by using a stylus pen or a part of a user's body. As an example, the display 170 may output a user interface configured to display the one or more physiological measurements, the one or more physiological parameters, the one or more physiological attributes, and/or the one or more medical conditions. For example, the display 170 may display a visual notification of, or associated with, the one or more physiological measurements, the one or more physiological parameters, the one or more physiological attributes, and/or the one or more medical conditions.

The communication interface 170 may establish, for example, communication between the device 101 and an external device (e.g., an electronic device 102 or a server 106). For example, the communication interface 170 may communicate with the external device (e.g., the electronic device 102 or the server 106) by being connected to a network 162 through wireless communication or wired communication. For example, as a cellular communication protocol, the wireless communication may use at least one of Long-Term Evolution (LTE), LTE Advance (LTE-A), Code Division Multiple Access (CDMA), Wideband CDMA (WCDMA), Universal Mobile Telecommunications System (UMTS), Wireless Broadband (WiBro), Global System for Mobile Communications (GSM), and the like. In an example, the network 162 may include at least one of a telecommunications network, a computer network (e.g., LAN or WAN), the internet, and a telephone network.

In addition, the communication interface 180 may communicate with the external device (e.g., the electronic device 102 and/or the server 106) via a communication connection 164 such as a wireless communication and/or wired communication. The wireless communication may include, for example, a near-distance communication 164. The near-distance communications 164 may include, for example, at least one of Wireless Fidelity (WiFi), Bluetooth, Near Field Communication (NFC), Global Navigation Satellite System (GNSS), and the like. According to a usage region or a bandwidth or the like, the GNSS may include, for example, at least one of Global Positioning System (GPS), Global Navigation Satellite System (Glonass), Beidou Navigation Satellite System (hereinafter, “Beidou”), Galileo, the European global satellite-based navigation system, and the like. Hereinafter, the “GPS” and the “GNSS” may be used interchangeably in the present document. The wired communication may include, for example, at least one of Universal Serial Bus (USB), High Definition Multimedia Interface (HDMI), Recommended Standard-232 (RS-232), power-line communication, Plain Old Telephone Service (POTS), and the like.

The server 106 may include a group of one or more servers. In an example, all or some of the operations executed by the device 101 may be executed in a different one or a plurality of electronic devices (e.g., the electronic device 102 and/or the server 106). In an example, if the device 101 needs to perform a certain function or service either automatically or based on a request, the device 101 may request at least some parts of functions related thereto alternatively or additionally to a different electronic device (e.g., the electronic device 102 and/or the server 106) instead of executing the function or the service autonomously. The different electronic devices (e.g., the electronic device 102 and/or the server 106) may execute the requested function or additional function, and may deliver a result thereof to the device 101. In one example, the electronic device 102 may comprise one or more complex programmable logic devices (CPLDs) or other programmable logic devices. In another example, the electronic device 102 may comprise a smart phone, a mobile device, a tablet computing device, a laptop computing device, a smartwatch, and the like. The electronic device 102 may be configured to process the one or more physiological measurements to determine the one or more physiological parameters. For example, the electronic device 102 may be configured to receive the one or more physiological measurements from the device 101, wherein the electronic device 102 may process the one or more physiological measurements to determine the one or more physiological parameters. For example, the electronic device 102 may determine the one or more physiological parameters based on applying the one or more physiological measurements to a multi-compartment model. The electronic device 102 may send the one or more physiological parameters to the device 101, wherein the device 101 may determine the one or more medical conditions based on the one or more physiological parameters and provide, or cause, a treatment associated with the one or more medical conditions. As a further example, the device 101 may send the determined one or more physiological parameters to the electronic device 102, wherein the electronic device 102 may be configured to determine the one or more medical conditions based on the one or more physiological parameters and send a treatment recommendation associated with the one or more medical conditions to the device 101. As a further example, the server 106 may be configured to receive the one or more physiological measurements from the device 101 and process the one or more physiological measurements to determine the one or more physiological parameters. As a further example, the device 101 may send the determined one or more physiological parameters to the server 106, wherein the server 106 may be configured to determine the one or more medical conditions based on the one or more physiological parameters and send a recommendation of a treatment associated with the one or more medical conditions to the device 101. The device 101 may provide the requested function or service either directly or by additionally processing the received result. For example, a cloud computing, distributed computing, or client-server computing technique may be used.

Each of the constitutional elements described in the present document may consist of one or more components, and names thereof may vary depending on a type of an electronic device. The device 101 may include at least one of the constitutional elements described in the present document. Some of the constitutional elements may be omitted, or additional other constitutional elements may be further included. Further, some of the constitutional elements of the device 101 according to various exemplary embodiments may be combined and constructed as one entity, so as to equally perform functions of corresponding constitutional elements before combination.

As a further example, one or more devices (e.g., device 101, electronic device 102, or server 106) may be configured to determine the one or more physiological parameters associated with an individual/patient such as an interstitial insulin elimination rate (αᵢ), a hepatic insulin elimination rate (αₕ), a renal insulin elimination rate (αₖ), or a permeability constant related to insulin diffusion (k). The physiological parameters are important for predicting one or more physiological attributes associated with an individual, such as a concentration of insulin in one or more of a vascular volume, an interstitial volume, a hepatic volume, or a renal volume. For example, the one or more devices may determine one or more physiological measurements associated with an individual. The one or more physiological measurements may comprise one or more of plasma insulin concentrations, a plasma volume, a vascular volume, an interstitial volume, a hepatic volume, or a renal volume. The one or more physiological measurements may be applied to a multi-compartment. The multi-compartment model may represent a bidirectional flux by diffusion of a hormone between a vascular compartment, an interstitial compartment, a hepatic compartment, and a renal compartment of the individual. For example, the multi-compartment model may comprise four non-linear differential equations. The hormone may comprise one or more of insulin, C-peptide, glucagon, amylin, somatostatin, insulin-like growth factors (IGFs), or incretins. The four non-linear differential equations may represent time-varying concentrations of insulin in a vascular plasma volume, in an interstitial volume, in a hepatic volume, and in a renal volume.

As an example, the differential equations may be presented in terms of flow rates. The flow rates facilitate the expression of the multi-compartment model in order to account for a fractional flow of plasma (and insulin) through the liver. The functions for plasma insulin (Iₚ(t)), interstitial insulin (Iᵢ(t)), hepatic insulin (Iₕ(t)) and renal insulin (Iₖ(t)) may be defined in concentration units (pmoles/L) and may define the multi-compartmental model. For example, the four non-linear differential equations may be expressed as:

$$V \frac{{dI}_{p} ( t )}{dt} = Z ( t ) - ( HPF - KPF ) * {I}_{p} ( t ) - k ( {I}_{p} ( t ) - {I}_{i} ( t ) ) + HPF * {I}_{h} ( t ) + KPF * {I}_{k} ( t ) , \qquad 1 )$$

$${V}_{i} \frac{{dI}_{i} ( t )}{dt} = + k ( {I}_{p} ( t ) - {I}_{i} ( t ) ) - {α}_{i} {V}_{i} * {I}_{i} ( t ) , \qquad 2 )$$

$${V}_{h} \frac{{dI}_{h} ( t )}{dt} = {Z}_{β} ( t ) + HPF * {I}_{p} ( t ) - HPF * {I}_{h} ( t ) - {α}_{h} {V}_{h} * {I}_{h} ( t ) , \qquad 3 )$$

$${V}_{k} \frac{{dI}_{k} ( t )}{dt} = + KPF * {I}_{p} ( t ) - KPF * {I}_{k} ( t ) - {α}_{k} {V}_{k} * {I}_{k} ( t ) , \qquad 4 )$$

where,
Iₚ, Iᵢ, Iₙ, Iₖ are plasma insulin concentrations (in units of picomoles/liter) in the four compartments representing vascular, interstitial, hepatic, and kidneys, respectively;
Vₚ, Vᵢ, Vₕ, Vₖ as the corresponding plasma volumes (units L);
Z(t) refers to exogenous insulin appearance (in units of insulin flow, pmol/minute) due to external infusion of insulin into the vascular compartment (V);
Z_{β} is the endogenous β-cell secretion (flow) rate of insulin from the pancreas into the portal vein (units pmol/min);
HPF, KPF are the fractional blood (plasma) flow rates (units L/min) in/out of liver and kidneys, respectively;
k is the permeability constant for insulin diffusion (in Fick's law) between vascular and interstitial compartments (units L/min); and
αᵢ, αₕ, αₖ are elimination rate constants (units l/min) from interstitial, hepatic, and kidney compartments, respectively.

The format of equations (1)-(4) emphasize that terms leaving one compartment should match the terms coming into another compartment. Elimination rate constants are likely the composition of (i) insulin binding to cell surface insulin receptor (IR) and (ii) subsequent internalization and elimination of the insulin. The terms αᵢVᵢ, αₕVₕ, αₖVₖ have units of L/min and are equivalent to the concept of clearance rates. Clearance rates depend on volumes and vary between individuals in an otherwise homogeneous group. In an example, the four differential equations may be utilized to determine elimination rates of C-peptide in the vascular volume, the interstitial volume, the hepatic volume, and the renal volume.

In an example, equations (1)-(4) may be converted to equations in concentrations by dividing by respective compartment volumes:

$$\frac{{dI}_{p} ( t )}{dt} = \frac{Z ( t )}{V} - ( \frac{HPF}{V} + \frac{KPF}{V} ) * {I}_{p} ( t ) - \frac{k}{V} ( {I}_{p} ( t ) - {I}_{i} ( t ) ) + \frac{HPF}{V} * {I}_{h} ( t ) + \frac{KPF}{V} * {I}_{k} ( t ) , \qquad 1 ’ )$$

$$\frac{{dI}_{i} ( t )}{dt} = + \frac{k}{{V}_{i}} ( {I}_{p} ( t ) - {I}_{i} ( t ) ) - {α}_{i} * {I}_{i} ( t ) , \qquad 2 ’ )$$

$$\frac{{dI}_{h} ( t )}{dt} = \frac{{Z}_{β} ( t )}{{V}_{h}} + \frac{HPF}{{V}_{h}} * {I}_{p} ( t ) - \frac{HPF}{{V}_{h}} * {I}_{h} ( t ) - {α}_{h} * {I}_{h} ( t ) , and \qquad 3 ’ )$$

$$\frac{{dI}_{k} ( t )}{dt} = + \frac{KPF}{{V}_{k}} * {I}_{p} ( t ) - \frac{KPF}{{V}_{k}} * {I}_{k} ( t ) - {α}_{k} * {I}_{k} ( t ) . \qquad 4 ’ )$$

The conversion between in vivo flow rate models and in vivo concentration models provides several advantages. For example, the advantages of the concentration models are: (i) that it is more closely related to the classical compartment models about the flow of mass between compartments governed by rate constants in units of 1/time (e.g., a 2-compartment model represents the vascular C-peptide concentration as a function of time C(t) and the extra-vascular concentration Y(t) in units of mass/volume with rate constants k1: C→Y and k2: Y→C both in units of l/minutes); (ii) measurements of vascular compounds are almost always in units of concentration and proposed models need to be compared to those values; and (iii) modeling in concentrations lead to an elimination expressed as a rate constant in units of l/min rather than the flow rate concept of clearance, wherein clearance may be expressed as CL=α*V as a flow rate (L/min) in flow rate models, which shows clearance depends on the volume being cleared and CL varies between individuals and within an otherwise homogeneous group, whereas the corresponding rate constant a varies less. For example, the advantages of flow models are: (i) terms in one equation match the term in another, which is valid for flow rate terms but not for concentration terms; and (ii) many physiological mechanisms are more easily expressed in flow rate terms.

As an example, the elimination may be a linear (e.g., proportional) function of concentration. For example, the linear term elimination term in the differential equations (α×concentration) becomes a monoexponential decay function in the solution of the differential equation. As such, the simple term(s) α*I(t) may replaced by Michaelis-Menten terms

$$\frac{{V}_{max} I ( t )}{{K}_{m} + I ( t )}$$

to represent saturable elimination.

As an example, delivery of insulin to the liver involves two types of flow rates to the liver: (i) the Plasma flow (L/min); and (ii) the insulin flow (pmoles/min). The connection between these two flow rates is insulin concentration and is an example of the universal law: plasma insulin flow=plasma flow x insulin concentration, where blood plasma flow=(1−0.45)*(whole) blood flow, which is a hematocrit calculation. An important application of this universal law can be written for elimination: clearance a*V is a plasma flow rate (e.g., the flow rate at which volume V is being cleared), and the model term a*V*Insulin concentration is a plasma insulin flow rate out of the system (e.g., insulin elimination) in units of pmol/min. For example, units of the plasma insulin flow rate are (L/min)*(pmoles/L)=(pmoles/min) and concentration Iₚ(t) is measured in the plasma. In addition, there are also fractional flows from the hepatic artery and portal vein to the liver. The delivery of insulin to the liver may be represented by combining the hepatic artery and portal venous sources of insulin into one systemic flow (HPF, pmoles/min) with concentration Iₕ(t). Thus, the fractional flows to the liver may be simplified and the input systemic flow rate is the same as the output flow rate in the hepatic veins (e.g., 1600 mL/min for blood and about 55%=100%-45% hematocrit of this value for plasma flow HPF≈880 mL/min).

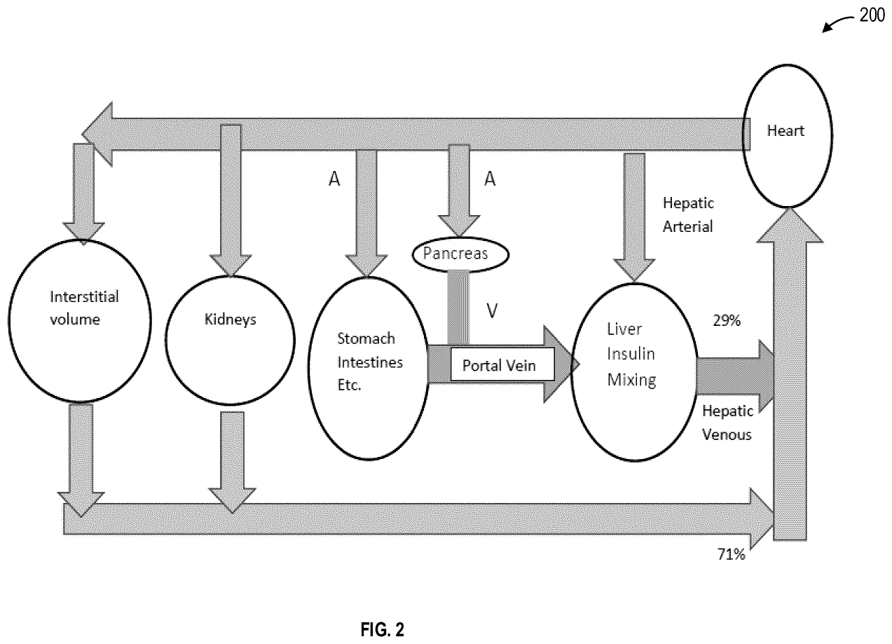

As an example, as shown in FIG. 2, blood plasma flow between the different compartments may involve six principles. The six principles about blood plasma flow may be utilized to simplify the construction of the compartmental model of insulin disposition in the human body. First, plasma flow is directly related to blood flow by hematocrit (%). Blood plasma is part of whole blood and equals whole blood minus the red blood cells, whose proportion (in volume) is measured by hematocrit. If hematocrit were 45% then plasma=(1−0.45)*(whole blood) and plasma flow=(1−0.45)*(blood flow).

Second, in addition to plasma flow, there is the plasma insulin flow. Insulin is considered to be carried in the plasma. The connection between these two flow rates is the plasma concentration of insulin I(t): plasma flow*I(t)=insulin flow.

Third, it may be useful to distinguish hepatic and extrahepatic plasma flows. The hepatic flow is represented by splanchnic circulation that includes portal and hepatic arterial flow to the liver and commensurate flow out of the liver (hepatic vein). A key feature is that insulin produced by the pancreas is secreted into the portal circulation. Extrahepatic blood plasma flow includes fractional systemic blood/plasma flow to tissues such as muscle, fat, kidneys, brain, etc. representing a dominant fraction of the total systemic blood flow. If hepatic blood flow=1.6 L/min and the total cardiac output=5.5 L/min, then the fraction of extrahepatic blood=1−(1.6/5.5)=1−0.29=0.71. In an example, the extrahepatic plasma is also 71% of the total plasma flow from the heart.

Fourth, the hepatic flow rate (HPF) requires special attention. The insulin in the blood plasma flow in the hepatic artery and the portal vein, and then is mixed in the liver sinusoids. An intermediate mixing also takes place in for the blood flow from the pancreas (e.g., B-cell secretion) and from the systemic blood flow in the portal vein (e.g., part of the system blood flow that also contains insulin I(t) in the plasma). As an example, the central compartment plasma flow through the liver (HPF) may be akin to the flow of plasma by the output to the hepatic vein. For the insulin flow through the liver, the inputs are from two sources: (i) insulin from the systemic plasma flow and (ii) Z_{β}=insulin secretion rate from B-cells in the pancreas (a flow rate). The first source is the sum of insulin from the hepatic artery plus the return of the systemic insulin through the portal vein, both of which are mixed in sinusoids in the liver. Using the plasma concentration of insulin equation above, HPF*Iₚ(t)+Z_{β}(t).

Fifth, there is a final mixing that takes place as the liver output plasma flow HPF*Iₕ(t), where Iₕ(t) is insulin concentration in the liver after hepatic elimination, which flow out of the liver through the hepatic vein then mixes with the extrahepatic plasma flow. For insulin flow, this gives HPF*Iₕ(t)+ (TPF−HPF)*Iₚ(t)=TPF*(0.29 Iₕ(t)+0.71Iₚ(t)), which in some form becomes the input of insulin to the vascular compartment, where it is typically measured. The output term HPF*Iₕ(t) from the liver contains the effect of both Z_{β}(t) and elimination, wherein the input to the liver is HPF*Iₚ(t)+Z_{β}(t). To compute the effect of Z_{β}(t) and elimination, the difference (e.g., output minus input) may be computed. For example, if Z_{β}(t) and elimination are both set to zero, then the input is HPF*Iₚ(t), and an effect and the output are the same. Thus, the effect of Z_{β}(t) and elimination is equal to HPF*Iₕ(t)−HPF*Iₚ(t)=HPF*(Iₕ(t)−Iₚ(t)), which is the change to be added to the insulin compartment. This informs the measurement of the arterio-venous differences in concentrations of various compounds, such as insulin, along with determinations of organ-specific flow rates.

Lastly, the left hand side differential equations (1)-(4) are the rates of change of plasma insulin (insulin flow rates). The right hand side of the model equations are constructed using five kinds of building blocks, wherein each represents changes in insulin flow rates. Diffusion (e.g., difference in concentrations (I(t)−Iᵢ(t)) comes from Fick's law for diffusion where the constant k is considered a flow rate (e.g., the plasma flow between the vascular and interstitial compartments). Blood/plasma flow through organ (e.g., difference in concentrations Iₕ(t)−Iₚ(t)) represents the effect on insulin due as the blood/plasma flows through the liver, Iₚ(t) is the input to, and Iₕ(t) is the output from, the liver. However, just ±Iₕ(t) is often in the model if the input Iₚ(t) has already been included. Infusion (e.g., appearance rates), which in the case of insulin may include endogenous insulin secretion by the B-cell, intravenous bolus or continuous infusion of exogenous insulin into the vascular compartment, or subcutaneous administration of exogenous insulin to the interstitial compartment and subsequent appearance in the vascular compartment by diffusion. The first order elimination of concentration (α) has a rate constant (units=l/min). However, in the flow model, α*V is expressed as clearance (L/min). Saturable elimination using a Michaelis-Menten function in the flow model is expressed as Vₘₐₓ in units of flow rate (L/min). As an example, the Michaelis parameter Km becomes HPF*Km in the flow model. Elimination represents a change in insulin mass due to (irreversible) removal of insulin outside the compartmental system. Renal clearance is considered as a separate compartment. In an example, most renal clearance is mediated through IR-dependent elimination of interstitial insulin, and therefore included in equation (1) above with Vₘₐₓ being utilized to account for variations related to renal elimination of insulin in the urine. The problem that the flow rate through the kidneys is fractional can be absorbed into Vₘₐₓ.

As an example, steady state solutions of the four compartmental concentrations may be obtained by setting the left hand side of the four derivatives of equations (1)-(4) equal to zero and using algebra, which may be expressed as a 4×4 matrix equation. Assuming Z and Z_{β} are known constant infusions, the steady state values of Iₚ, Iᵢ, Iₕ and Iₖ may be obtained by solving the following four equations:

$$[ \begin{matrix}k + ( HPF + KPF ) & - k & - HPF & - KPF \\ - k & k + {α}_{i} {V}_{i} & 0 & 0 \\ - HPF & 0 & HPF + {α}_{h} {V}_{h} & 0 \\ - KPF & 0 & 0 & KPF + {α}_{k} {V}_{k}\end{matrix} ] [ \begin{matrix}{I}_{p} \\ {I}_{i} \\ {I}_{h} \\ {I}_{k}\end{matrix} ] = [ \begin{matrix}Z \\ 0 \\ {Z}_{β} \\ 0\end{matrix} ] , \qquad ( 5 )$$

wherein rows 2 and 4 of the above matrix provide Iᵢ=a*Iₚ and Iₖ=dIₚ, where

$$a = \frac{k}{( k + {α}_{i} {V}_{i} )} and d = \frac{K P F}{( K PF + {α}_{k} {V}_{k} )} ;$$

row 3 provides

$${I}_{h} = {bI}_{p} + \frac{{Z}_{β}}{H PF + {α}_{h} {V}_{h}} = {bI}_{p} + \frac{b * {Z}_{β}}{H P F} ,$$

where

$$b = \frac{H P F}{( KPF + {α}_{h} {V}_{h} )} ;$$

and row 1 provides (k+(HPF+KPF))Iₚ=kIᵢ+HPFIₖ+Z or substituting for Iₕ and Iₖ,

$$( k + ( H P F + K P F ) ) {I}_{p} = ka * {I}_{p i} + HPF ( {bI}_{p} + \frac{b * {Z}_{β}}{H P F} ) + {KPFdI}_{p} + Z or {cI}_{p} = Z + b * {Z}_{β} ,$$

where c=k−ka+(HPF+KPF)−HPF*b−KPF*d or c=k(1−a)+HPF(1−b)+KPF(1−d). Thus,

$$5 ’ ) \{ \begin{matrix}{I}_{p} = \frac{Z + b ⋆ {Z}_{β}}{c} and \\ {I}_{i} = a ⋆ {I}_{p} and \\ {I}_{h} = b ⋆ {I}_{p} + \frac{b ⋆ {Z}_{β}}{H P F} and \\ {I}_{k} = d ⋆ {I}_{p}\end{matrix} ,$$

wherein the factors are the reduction of Iₚ to

$${I}_{i} = a = \frac{k}{( k + {α}_{i} * {V}_{i} )} < 1 ,$$

and the reduction of secretion Z_{β} in the

$$liver = b = \frac{H P F}{HPF + {α}_{h} * {V}_{h}} < 1 ,$$

and reduction of Iₚ to

$${I}_{k} = d = \frac{KPF}{( KPF + {α}_{K} * {V}_{K} )} ,$$

and the fractions of circulating Iₚ related to the interstitial, liver and kidneys=c=k(1−a)+HPF(1−b)+KPF(1−d), wherein c>0 and all steady state concentrations are positive.

As an example, the steady state solutions imply some order relationships between the steady state solutions such as Iᵢ<Iₚ and I<Iₚ since a<1 and d<1, respectively. For example, Z=HPF*an insulin concentration such that the expression

$$\frac{{Z}_{β}}{H P F}$$

comprises an insulin concentration. In addition,

$$b = \frac{H P F}{HPF + {α}_{h} * {V}_{h}} = \frac{1}{1 + \frac{1}{HPF / {a}_{h} * {V}_{h}}} ,$$

and αₕ*Vₕ comprises a plasma flow (e.g., clearance) such that HPF/αₕ*Vₕ comprises the ratio of two plasma flow rates (e.g., a number). As an example, HPF(1−b) comprises a flow rate and c comprises a flow rate. For example, the expression for Iₚ in equation (5′) comprises the ratio of insulin flow rate and plasma flow rate and as such comprises an insulin concentration. The comparison of Iₖ and Iᵢ depend on the comparison of d and a; that is, the comparison of HPF/αₖ*Vₖ and k/αᵢ*Vᵢ. Thus, HPF/αₖ*Vₖ<k/aᵢ*Vᵢ if Iₖ<Iᵢ. As an example, αᵢ*Vᵢ<KPF, implying

$$\frac{1}{2} < d < 1 and \frac{1}{2} {I}_{p} < {I}_{k} < {I}_{p} , and \frac{1}{2} {I}_{p} < {I}_{i} < {I}_{p} .$$

In addition, Iₕ<Iₚ+Z_{β}/HFP since b<1. Without exogenous insulin input Z=0, Iₚ is proportional to

$${Z}_{β} : {I}_{p} = \frac{b}{c} {Z}_{β}$$

in steady state. As an example, for steady state for a constant exogenous insulin input (Z),

$$[ {I}_{p} - {I}_{p} ( 0 ) ] = \frac{Z}{c} .$$

Multiple considerations may be taken into account in constructing the compartmental model: (i) (TBF−HPF)*Iₚ is the plasma insulin flow in the vascular plasma volume (Vₚ); (ii) Iₚ in Vₚ is in a diffusion relationship with insulin in the interstitial volume Vᵢ, wherein the diffusion contribution to Vᵢ is governed by Fick's law and may be expressed by the term +k(Iₚ−Iᵢ) which subtracted from the vascular volume Vₚ; (iii) insulin elimination from the interstitial volume Vᵢ equal to −αᵢ*Vᵢ*Iᵢ and the insulin elimination from the renal volume Vₖ is −αₖ*Vₖ*Iₖ; (iv) HPF*Iₚ is the input to the liver compartment with volume Vₕ via the hepatic artery and portal vein, wherein the input to the renal compartment is KPF*Iₚ; (v) the input Z_{β} from the B cells in the pancreas that reach the liver compartment with volume Vₕ via the portal vein, and thus, the delivery of insulin to Vₚ is HPF*Iₚ+Z_{b}, wherein HPF*Iₚ(t) in the delivery of insulin to the hepatic compartment is subtracted from the vascular compartment, wherein KPF*Iₚ(t) is the delivery of insulin to the renal compartment and is subtracted from the vascular compartment; (vi) insulin elimination from Vₕ:−αₕ*Vₕ*Iₕ, wherein the insulin concentration Iₕ is the result of a differential equation such as

$${V}_{h} \frac{{dI}_{h}}{dt} = HPF * {I}_{p} ( t ) + {Z}_{β} ( t ) - {α}_{h} * {V}_{h} * {I}_{h} ( t ) ;$$

and (vii) to balance the input insulin flow HPF*Iₚ, the output insulin flow HPF*Iₕ from Vₕ is accounted for, wherein HPF*Iₚ merges with (TBF−HPF)*Iₚ at the junction of the hepatic vein and inferior vena cava, which equals TBF*Iₚ+HPF(Iₕ−Iₚ) and the term HPF(Iₕ−Iₚ) is the change in insulin flow (quantity/time) feeding back to the vascular volume Vₚ. As such, the terms −k(Iₚ(t)−Iᵢ(t)) from (ii), +HPF*(Iₕ(t)−Iₚ(t)) from (vii), −HPF*Iₚ(t) from (v), and +Z(t) representing the infusion of exogenous insulin into the vascular volume (Vₚ) may be used to construct the first non-linear differential equation; the terms +k(Iₚ(t)−Iᵢ(t)) from (vii) and −αᵢVᵢ*Iᵢ(t) from (iii) may be used to construct the second non-linear differential equation; the terms +HPF*Iₚ(t)+Z_{β}(t) from (v), −αₕ*Vₕ*Iₕ(t) from (vi), and −HPF*(Iₕ(t)−Iₚ(t)) from (vii) may be used to construct the third non-linear differential equation; and the terms +KPF*Iₚ(t) from (v), +KPF*(Iₖ(t)−Iₚ(t)) from (vii), and −αₖ*Vₖ*Iₖ(t) from (iii) may be used to construct the fourth non-linear differential equation.

Convolution integral solutions may be applied to each of the equations (1′)-(4′), wherein equations (2′)-(4′) have solutions in terms of Iₚ(t). However, the convolution integral solution for Iₚ(t) is circular in equation (1′). As an example, the convolution solutions may give information about the average delay of Iᵢ, Iₕ, and Iₖ with respect to Iₚ. For example, the solution of equation (3′) comprises a convolution with exponential delay and input (HPF*Iₚ(t)+Z_{β}(t))/Vₕ. Thus, equation (3′) may be written in standard form \dot{y}+p(t)y=q(t), such that:

$$3 a ) \frac{{dI}_{h} ( t )}{dt} + ( \frac{H P F}{{V}_{h}} + {α}_{h} ) {I}_{h} ( t ) = \frac{( HPF * {I}_{p} ( t ) + {Z}_{β} ( t ) )}{{V}_{h}} .$$

wherein the general solution of the standard form using the integrating factor e^{P(t)}, where

$$P ( t ) = {\int }_{0}^{t} p ( τ ) d τ ,$$

is

$$y ( t ) = {e}^{- P ( t )} y ( 0 ) + {e}^{- P ( t )} {\int }_{0}^{t} {e}^{P ( τ )} q ( τ ) d τ = {e}^{- P ( t )} \{ y ( 0 ) + {\int }_{0}^{t} {e}^{+ P ( τ )} q ( τ ) d τ \} .$$

Thus, the solution Iₕ(t) comprises a convolution integral plus a decaying initial condition, such that:

$${I}_{h} ( t ) = {I}_{h} ( 0 ) {e}^{- β t} + {\int }_{0}^{t} {e}^{- β ( t - τ )} \{ \frac{HPF * {I}_{p} ( τ ) + {Z}_{β} ( τ )}{{V}_{h}} \} d τ , where β = ( \frac{H P F}{{V}_{h}} + {α}_{h} ) . \qquad 3 ” )$$

In an example, if Z_{β}(t)=0 and the input Iₚ(0) is stable before the B-cells are simulated to produce insulin, Iₕ(0)=Iₚ(0). For equation (2′), the convolution integral may be expressed as:

$${I}_{i} ( t ) = {I}_{i} ( 0 ) {e}^{- β t} + {\int }_{0}^{t} {e}^{- β ( t - τ )} \{ \frac{{I}_{p} ( τ )}{{V}_{i}} \} d τ , where β = ( \frac{k}{{V}_{i}} + {α}_{i} ) . \qquad 2 ” )$$

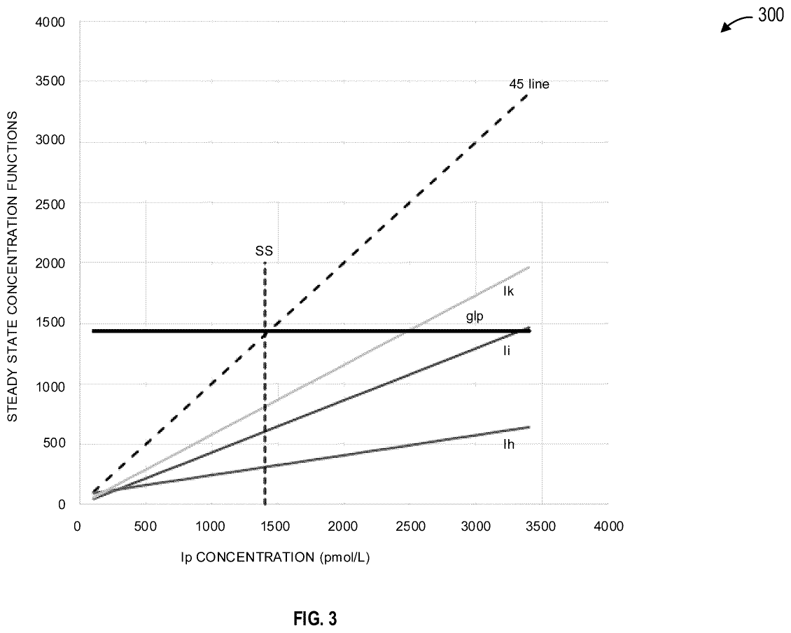

FIG. 3 shows an example graphical solution/algorithm 300 for steady state solutions of equation (5′). As shown in FIG. 3, the x-axis represents varying values of Iₚ given by the first row of equation (5′) obtained by varying Z and Z_{β}. The values of Iᵢ and Iₖ are represented by lines through the origin and Iₕ comprises a line with a positive intercept depending on Z_{β}. For given values of Z and Z_{β}, the steady state value of Iₚ in the first row of equation (5′) is considered a constant (e.g., horizontal line as shown in FIG. 3). The steady state Iₚ is represented by the intersection of the horizontal solid line and the identity line (e.g., diagonal dashed line). Thus, the steady state solutions for Iᵢ, Iₖ, and Iₕ on their respective lines are represented by their intersection with the vertical dashed line (corresponding to the steady state value of Iₚ).

In an example, before replacing the first order elimination α*I

$$( e . g . , units of \frac{1}{min} * \frac{pmole}{L} )$$

by Michaelis-Menten like elimination

$$\frac{{V}_{max} ⋆ I ( t )}{{K}_{m} + I ( t )}$$

in the four non-linear differential equations (1′)-(4′), the equations may be re-parameterized by setting

$${α}^{′} = \frac{{V}_{max}}{{K}_{m}}$$

(e.g., with units

$$\frac{1}{min}$$

where Vmax is in units of

$$( \frac{p mol}{L} ) / min$$

and Michaelis constant Kₘ in units of concentration pmol/L). Thus, the

$$saturable eleimination term = {α}^{′} * \frac{{K}_{m} * I ( t )}{{K}_{m} + I ( t )} ,$$

wherein the effective elimination rate

$${α}_{eff} = {α}^{′} \frac{{K}_{m}}{{K}_{m} + I ( t )} .$$

Thus, α_{eff}<α′ for all I(t), but for small I(t), effective alpha is approximately constant α_{eff}=α′ (e.g., α_{eff} approaches α′ as I(t) approaches 0). In one example, for I(t) small, Michaelis-Menten elimination is approximately first order elimination α′*I. Also for I(t)=Kₘ,

$${α}_{eff} = \frac{{a}^{′}}{2} .$$

In anther example, for I(t) large, the effective alpha α_{eff} is small (e.g., approaches 0). For example, the Michaelis-Menten like function approaches an asymptote Vₘₐₓ as I(t) becomes large and becomes 0th order elimination. As such, a constant portion of insulin concentration (=Vₘₐₓ(pmol/L)/min) is eliminated for each unit of time (e.g., minutes, seconds, hours, etc.).

The reparameterization of Michaelis-Menten elimination may also be expressed as

$${α}^{′} * \frac{1}{2} H ( I ( t ) , {K}_{m} ) .$$

Thus, the system of differential equations with Michael-Menten elimination representing saturable elimination may be written using substitution such that equations (1′)-(4′) may be expressed for saturable elimination as:

$$\frac{{dI}_{p} ( t )}{dt} = \frac{Z ( t )}{V} - ( \frac{H P F}{V} + \frac{K P F}{V} ) * {I}_{p} ( t ) - \frac{k}{V} ( {I}_{p} ( t ) - {I}_{i} ( t ) ) + \frac{H P F}{V} * {I}_{h} ( t ) + \frac{K P F}{V} * {I}_{k} ( t ) , \qquad 1 ’ )$$

$$\frac{{dI}_{i} ( t )}{dt} = + \frac{k}{{V}_{i}} ( {I}_{p} ( t ) - {I}_{i} ( t ) ) - {α}_{i}^{′} * {I}_{i} ( t ) , where {α}_{i}^{′} = \frac{{V}_{max ( i )}}{{K}_{i}} , \qquad 2 ” )$$

$$\frac{{dI}_{h} ( t )}{dt} = \frac{{Z}_{β} ( t )}{{V}_{h}} + \frac{H P F}{{V}_{h}} * {I}_{p} ( t ) - \frac{H P F}{{V}_{h}} * {I}_{h} ( t ) - {α}_{h}^{′} * {I}_{h} ( t ) , where {α}_{h}^{′} = \frac{{V}_{max ( h )}}{{K}_{h}} , \qquad 3 ” )$$

$$\frac{{dI}_{k} ( t )}{dt} = + \frac{K P F}{{V}_{k}} * {I}_{p} ( t ) - \frac{K P F}{{V}_{k}} * {I}_{k} ( t ) - {α}_{k}^{′} * {I}_{k} ( t ) , where {α}_{k}^{′} = \frac{{V}_{max ( k )}}{{K}_{k}} . \qquad 4 ” )$$

Equation (1′) (e.g., central/vascular plasma compartment) is unchanged as it does not contain an elimination term. In an example, an approximation for the combined elimination term by kidney is

$$A * {α}_{k} * {I}_{k} + ( 1 - A ) * {α}_{k}^{′} \frac{{K}_{k}}{{K}_{k} + {I}_{k}} * {I}_{k} ,$$

where A=0.6.

As an example, in order to apply Michaelis-Menten elimination to the saturable elimination of insulin, the elimination term αᵢ*Iᵢ(t) in equation (2′) may be replaced using the concentration Iᵢ(t) to obtain the elimination term

$$\frac{{V}_{max} * {I}_{i} ( t )}{{K}_{m} + {I}_{i} ( t )} = {α}^{′} \frac{{K}_{m} {V}_{h}}{{K}_{m} + {I}_{i} ( t )} {I}_{i} ( t ) ,$$

where

$${α}^{′} = \frac{{V}_{max}}{{K}_{m} * {V}_{i}} .$$

The effective alpha is

$${α}_{eff} = {α}^{′} \frac{{K}_{m}}{{K}_{m} + {I}_{i} ( t )} ,$$

which is equal to

$$\frac{α′}{2} at {I}_{h} = {K}_{m} .$$

As an example, similar replacements may be applied to equations (3′) and (4′). As such, the differential equation of the resulting hepatic insulin flow rate Iₕ(t) may become:

$${V}_{h} \frac{{dI}_{h} ( t )}{dt} = {Z}_{β} ( t ) + HPF * {I}_{p} ( t ) - HPF * {I}_{h} ( t ) - {{α}^{′}}_{h} \frac{{K}_{m} {V}_{h}}{{K}_{m} + {I}_{i} ( t )} {I}_{i} ( t ) . \qquad 3 )$$

As an example, if Iₚ is measured in steady state, the reduction factor in a steady state equation Iᵢ=a Iₚ in equation (5′) may become

$$a = k / ( k + {{α}^{′}}_{1} \frac{{K}_{1} {V}_{1}}{{K}_{1} + {I}_{i}} ) ,$$

where

$${{α}^{′}}_{1} = \frac{{V}_{max 1}}{{K}_{1}}$$

and the Michaelis constant may be relabeled as K₁. As such collecting terms in Iᵢ and clearing functions,

$$( k + {{α}^{′}}_{1} \frac{{K}_{1} {V}_{1}}{{K}_{1} + {I}_{i}} ) {I}_{i} = {kI}_{p} or ( k ( {K}_{1} + {I}_{i} ) + {{α}^{′}}_{1} {K}_{1} {V}_{1} ) {I}_{i} = k ( {K}_{1} + {I}_{i} ) {I}_{p}$$

which is a quadratic equation

$${a}_{1} {I}_{i}^{2} + {b}_{1} {I}_{i} + {c}_{1} = 0 , \\ where {a}_{1} = k and {b}_{1} = k ( {K}_{1} - {I}_{p} ) + {{α}^{′}}_{1} {K}_{1} {V}_{1} and {c}_{1} = - {kK}_{1} {I}_{p}$$

As such,

$${I}_{i} = \frac{- b + \sqrt{{b}^{2} - 4 ac}}{2 a} = \frac{- {b}_{1} + \sqrt{{b}_{1}^{2} + 4 {k}^{2} {K}_{1} {I}_{p}}}{2 k} .$$

An analogous steady state solution Iₖ for the fourth equation of equation (5′) with reduction fraction d and the Michaelis-Menten like saturable elimination of insulin from the kidneys results in a quadratic equation with coefficients a₃=KPF and b₃=KPF(K₃−Iₚ)+α′₃K₃Vₖ and c₃=−KPF*K₃Iₚ.

For the steady state solution Iₕ, the third equation of equation (5′) may be rewritten as

$${I}_{h} = b ( {I}_{p} + \frac{{Z}_{β}}{HPF} ) .$$

The reduction fraction b is replaced by

$$( {I}_{p} + \frac{{Z}_{β}}{HPF} ) = \frac{HPF * {I}_{p} + {Z}_{β}}{HPF} .$$

Michaelis-Menten saturable elimination of insulin from the liver results in a quadratic equation with the coefficients

$${a}_{2} = HPF , {b}_{2} = HPF ( {K}_{2} - ( {I}_{p} + \frac{{Z}_{β}}{HPF} ) ) + {{α}^{′}}_{2} {K}_{2} {V}_{h} , \\ and {c}_{2} = - HPF * {K}_{2} ( {I}_{p} + \frac{{Z}_{β}}{HPF} ) .$$

The first equation of equation (5′) gives an equation that the steady state Iₚ must satisfy. The denominator c=k(1−a)+HPF(1−b)+KPF(1−d), which is a function of a, b, and d and which in turn are functions of Iₚ.

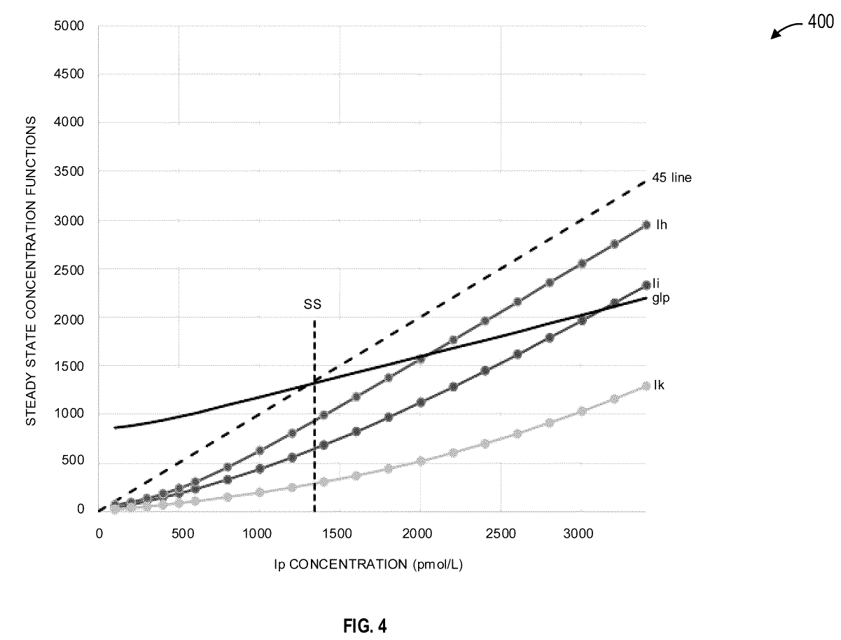

FIG. 4 shows a graphical solution 300 of all saturable elimination of insulin. Iᵢ, Iₖ, and Iₕ as may be computed as functions Iₚ. The left hand side of the first equation of

$${I}_{p} , g ( {I}_{p} ) = \frac{Z + b * {Z}_{β}}{c} ,$$

equation (5′) may be computed as a function of where g(Iₚ) intersects the 45 degree line Iₚ=Iₚ is the steady state solution for Iₚ. If all functions Iᵢ, Iₖ, Iₕ and g(Iₚ) versus Iₚ are placed on the same plot, the vertical line through the intersection also gives the rest of the compartmental steady state values as intersections.

In an example, measuring the C-peptide time series may provide a direct estimate of the B-cell activity and Z_{β}(t). C-peptide is a 31-amino acid polypeptide that is released into the blood by pancreatic beta cells in the same molar ratio as insulin. C-peptide levels can be measured in plasma and are often used to monitor beta cell function. Normal C-peptide levels in a healthy person are around 0.3-0.6 nanomoles per liter (nmol/l) when fasting and 1-3 nmol/l after a meal. High levels of C-peptide can indicate insulin resistance, insulinoma, or kidney disease, while low levels are often found in people with type 1 or type 2 diabetes.

A C-peptide multi-compartment model (e.g., 4-compartment model), for example, may be a nested, or a sub-model, of the insulin multi-compartment model (e.g., 4-compartment model). However, C-peptide is not eliminated from the liver but from the kidneys. Assuming there is no loss of C-peptide in the liver, the liver compartment may be pooled with the vascular compartment by summing C-peptide quantities V*Iₚ+Vₕ*Iₕ in the volume V+Vₕ. As an example, the resulting concentration may be relabeled (V*Iₚ+Vₕ*Iₕ)/(V+Vₕ) as C(t) and volume (V+Vₕ) as Vₚ and concentration Iᵢ as Y(t) and Z_{β}(t) as S (t) to obtain:

$${V}_{p} {C}^{′} ( t ) = - ( k + KPF ) * C ( t ) + k * Y ( t ) + KPF * {C}_{k} ( t ) + S ( t ) , \qquad 1 \text{*)}$$

$${V}_{i} * {Y}^{′} ( t ) = + k ( C ( t ) - Y ( t ) ) - {α}_{i} * {V}_{i} * Y ( t ) , and \qquad 2 \text{*)}$$

$${V}_{k} * {{C}^{′}}_{K} ( t ) = + KPF * ( C ( t ) - {C}_{k} ( t ) ) - {α}_{k} * {V}_{k} * {C}_{k} ( t ) . \qquad 3 \text{*)}$$

The implementation of the multi-compartmental model allows for estimating insulin disposition parameters (e.g., interstitial insulin elimination rates (αᵢ), hepatic insulin elimination rates (αₕ), renal insulin elimination rates (αₖ), or a permeability constant related to insulin diffusion (k)) based on utilizing an algebraic method (e.g., a convolution integral solution method) that bypasses the use of iterative, numerical integration methods. The use of convolution integral solutions may be applied to an implementation of individualized parameter solutions using hybrid closed-loop insulin delivery systems. Hybrid closed-loop insulin delivery systems have limited computational capacity, and thus, lack the ability to perform numerical integration. However, hybrid closed-loop insulin delivery systems may be able to determine relevant insulin disposition parameters by utilizing algebraic methods that involve the use of convolution integral solution methods.

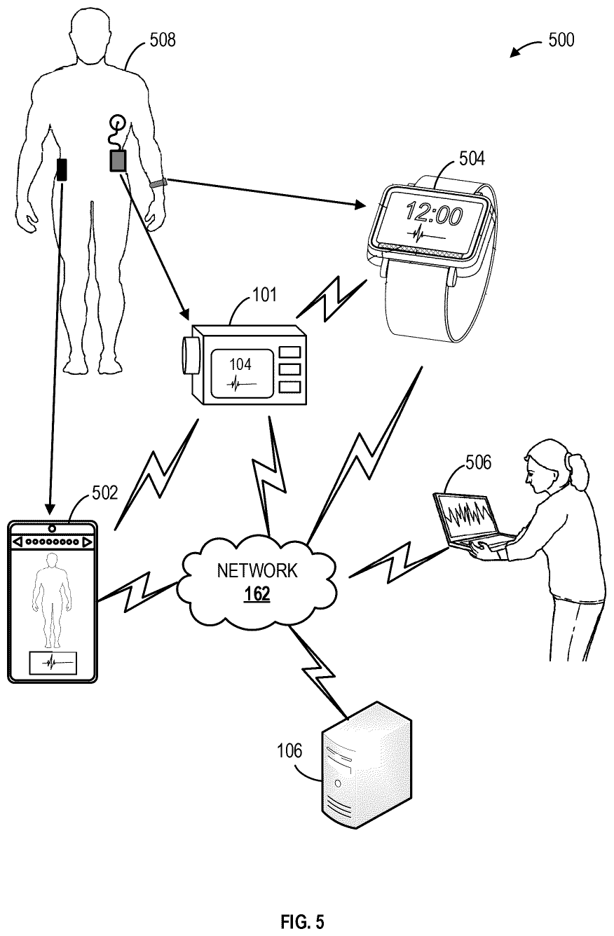

FIG. 5 shows an example system environment 500. As an example, a device 101, such as an insulin pump, may comprise a computing device configured to determine one or more physiological parameters of a human subject (e.g., patient, individual, etc.), such as an interstitial insulin elimination rate, a hepatic insulin elimination rate, a renal insulin elimination rate, and a permeability constant related to insulin diffusion. As an example, the device 101 may comprise, a mobile phone, a smart phone, a tablet computer, a laptop, a desktop computer, a smartwatch, a smart glass, an insulin pump device, and the like. As an example, the device 101 may comprise a computing device for controlling an insulin pump. As an example, the device 101 and the insulin pump may be integrated together as a single device or may comprise separate devices. The device 101 may be configured to control the insulin pump for delivering insulin to a human subject/individual via tubing connected between the insulin pump and an infusion set affixed/attached to a location of the individual's body.

The device 101 may be configured to process one or more physiological measurements associated with an individual and apply the physiological measurements to a multi-compartment model to determine the one or more physiological parameters associated with the individual. For example, the device 101 may include logic (e.g., hardware, software, firmware, etc.) that may be implemented to determine one or more physiological parameters of an individual.

For example, the device 101 may determine one or more physiological measurements associated with an individual. The one or more physiological measurements may comprise one or more of plasma insulin concentrations, a plasma volume, a vascular volume, an interstitial volume, a hepatic volume, or a renal volume. The device 101 may apply the one or more physiological measurements to a multi-compartment model that represents a bidirectional flux by diffusion of a hormone between a vascular compartment, an interstitial compartment, a hepatic compartment, and a renal compartment of the individual. For example, the multi-compartment model may comprise four non-linear differential equations that represent time-varying concentrations of insulin in a vascular plasma volume, in an interstitial volume, in a hepatic volume, and in a renal volume. The hormone may comprise one or more of insulin (e.g., endogenous and/or exogenous insulin), C-peptide, glucagon, amylin, somatostatin, insulin-like growth factors (IGFs), or incretins. The device 101 may determine one or more physiological parameters associated with the individual based on the application of the one or more physiological measurements to the multi-compartment model. The one or more physiological parameters may comprise one or more of an interstitial insulin elimination rate (αᵢ), a hepatic insulin elimination rate (αₕ), a renal insulin elimination rate (αₖ), or a permeability constant related to insulin diffusion (k). The device 101 may determine one or more medical conditions associated with the individual based on the one or more physiological parameters. The one or more medical conditions associated with the individual may comprise one or more of diabetes (e.g., type 1, 2, and/or 3c diabetes), insulin resistance, hypoglycemia, insulinoma, Cushing's syndrome, acromegaly, hypothyroidism, hypertension, dyslipidemia, hyperuricemia, or endothelial dysfunction In an example, the multi-compartmental model may be utilized to track/monitor endogenous insulin secretion and exogenous insulin administration in individuals/patients with preserved beta cell function (e.g., in individuals/patients with type 2 diabetes mellitus). In an example, measurements of insulin sensitivity and beta cell function may be utilized to predict a probability of future development of diabetes in an individual/patient. For example, the multi-compartmental model may be utilized to increase the accuracy of obtaining clinically useful measures of insulin sensitivity and insulin secretory capacity over other methods, such as HOMA-IR and HOMA-beta. In an example, the one or more medical conditions may be determined based on one or more physiological attributes associated with the individual. For example, the one or more physiological attributes may comprise a concentration of insulin in one or more of a vascular volume, an interstitial volume, a hepatic volume, or a renal volume. The one or more physiological attributes associated with the individual may be determined based on the one or more physiological parameters. As an example, the one or more medical conditions may be determined based on an appearance and elimination rate of the hormone in the individual. For example, the appearance and elimination rate of the hormone in the individual may be determined based on the one or more physiological parameters.

The device 101 may cause one or more treatments associated with the one or more medical conditions associated with the individual to be administered to the individual based on the one or more physiological parameters. As an example, the one or more treatments may be administered based on the one or more physiological attributes associated with the individual. As an example, the one or more treatments may be administered based on the appearance and elimination rate of the hormone in the individual. The one or more treatments may comprise administering the hormone, or hormone replacement therapy. In an example, the hormone may be administered to the individual when the one or more medical conditions associated with the individual are determined. The administration of the hormone may be adjusted based on the one or more physiological parameters. For example, an inflow of insulin to the individual may be reduced or increased based on the one or more physiological parameters.

As an example, the device 101 may be configured to communicate with one or more external devices (e.g., a mobile phone/device 502, a smartwatch 504, a laptop/tablet computer 506, and/or a server 106) via network 162 and/or via near-distance communication (e.g., NFC, Bluetooth, etc.). In example, one or more of the mobile phone/device 502 and the smartwatch 504 may be worn by a user (e.g., the individual), wherein the user may utilize the phone/device 502 and the smartwatch 504 to monitor one or more of the one or more physiological measurements, the one or more physiological parameters, the one or more physiological attributes, and/or the one or more medical conditions of the user. For example, the device 101 may output one or more of the one or more physiological measurements, the one or more physiological parameters, the one or more physiological attributes, and/or the one or more medical conditions to one or more of the mobile phone/device 502, the smartwatch 504, the laptop/tablet computer 506, and/or the server 106. In an example, one or more of the mobile phone/device 502, the smartwatch 504, and/or the laptop/tablet computer 506 may be configured to display one or more of the one or more physiological measurements, the one or more physiological parameters, the one or more physiological attributes, and/or the one or more medical conditions to a user 508. For example, one or more of the mobile phone/device 502, the smartwatch 504, and/or the laptop/tablet computer 506 may display a visual notification of, or associated with, the one or more physiological measurements, the one or more physiological parameters, the one or more physiological attributes, and/or the one or more medical conditions. In an example, one or more of the mobile phone/device 502, the smartwatch 504, the laptop/tablet computer 506 and/or the server 106 may be configured to process the one or more physiological measurements to determine the one or more medical conditions. One or more of the mobile phone/device 502, the smartwatch 504, the laptop/tablet computer 506 and/or the server 106 may output the one or more medical conditions to the device 101, wherein the device 101 may administer, or adjust, the treatment associated with the one or more medical conditions. In an example, one or more of the mobile phone/device 502, the smartwatch 504, the laptop/tablet computer 506 and/or the server 106 may output a signal to trigger the device 101 to administer, or adjust, the treatment associated with the one or more medical conditions.

In an example, the device 101 may comprise a hybrid closed-loop insulin pump, combining automated insulin delivery with one or more manual adjustments. For example, the hybrid closed-loop insulin pump may utilize a continuous glucose monitor (CGM) to track blood sugar levels and a control algorithm to automatically adjust basal insulin delivery, while requiring the user to manually enter information, such as meal boluses. For example, the multi-compartmental model may be utilized to infer insulin concentrations in relevant compartments based on a time series of interstitial glucose measurements obtained by monitoring glucose continuously. For example, the device 101 may utilize the insulin concentration calculations to automatically adjust the basal insulin delivery rate throughout the day and night in order to keep the user's blood sugar within a target range. The implementation of the multi-compartmental model allows for estimating insulin disposition parameters (e.g., interstitial insulin elimination rates (αᵢ), hepatic insulin elimination rates (αₕ), renal insulin elimination rates (αₖ), or a permeability constant related to insulin diffusion (k)) based on utilizing an algebraic method (e.g., a convolution integral solution method) that bypasses the use of iterative, numerical integration methods. The use of convolution integral solutions may be applied to an implementation of individualized parameter solutions using hybrid closed-loop insulin delivery systems. Hybrid closed-loop insulin delivery systems have limited computational capacity, and thus, lack the ability to perform numerical integration. However, hybrid closed-loop insulin delivery systems may be able to determine relevant insulin disposition parameters by utilizing algebraic methods that involve the use of convolution integral solution methods.

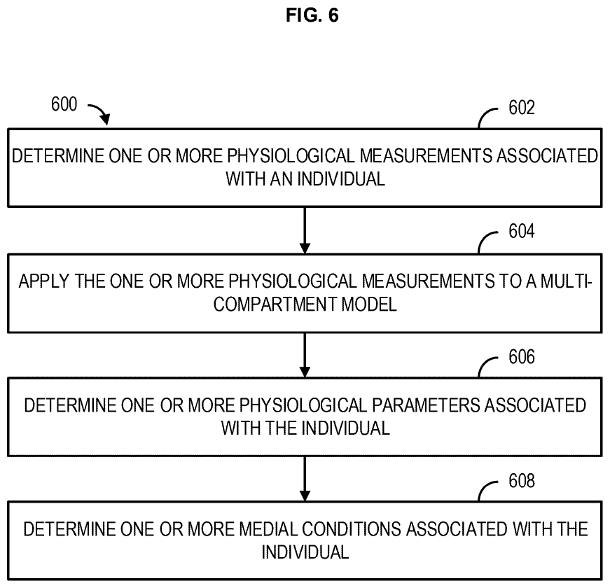

FIG. 6 shows an example method 600 for determining one or more physiological parameters associated with an individual based on one or more physiological measurements associated with the individual. Method 600 may be implemented by a computing device (e.g., device 101, electronic device 102, server 106, or any combination thereof). At step 602, one or more physiological measurements associated with an individual may be determined. For example, a computing device (e.g., device 101, electronic device 102, server 106, or any combination thereof) may determine the one or more physiological measurements associated with the individual. The one or more physiological measurements may comprise one or more of plasma insulin concentrations, a plasma volume, a vascular volume, an interstitial volume, a hepatic volume, or a renal volume.

At step 604, the one or more physiological measurements may be applied to a multi-compartment model. For example, the computing device (e.g., device 101, electronic device 102, server 106, or any combination thereof) may apply the one or more physiological measurements to the multi-compartment model. The multi-compartment model may represent a bidirectional flux by diffusion of a hormone between a vascular compartment, an interstitial compartment, a hepatic compartment, and a renal compartment of the individual. The hormone may comprise one or more of insulin, C-peptide, glucagon, amylin, somatostatin, insulin-like growth factors (IGFs), or incretins. The multi-compartment model may comprise four non-linear differential equations. The four non-linear differential equations may represent time-varying concentrations of insulin in a vascular plasma volume, in an interstitial volume, in a hepatic volume, and in a renal volume.

At step 606, one or more physiological parameters associated with the individual may be determined based on the application of the one or more physiological measurements to the multi-compartment model. For example, the computing device (e.g., device 101, electronic device 102, server 106, or any combination thereof) may determine the one or more physiological parameters associated with the individual based on the application of the one or more physiological measurements to the multi-compartment model. For example, the multi-compartment model may be solved, based on the one or more physiological measurements, for the one or more physiological parameters. The one or more physiological parameters may comprise one or more of an interstitial insulin elimination rate (αᵢ), a hepatic insulin elimination rate (αₕ), a renal insulin elimination rate (αₖ), or a permeability constant related to insulin diffusion (k).

At step 608, one or more medical conditions may be determined based on the one or more physiological parameters. For example, the computing device (e.g., device 101, electronic device 102, server 106, or any combination thereof) may determine the one or more medical conditions based on the one or more physiological parameters. The one or more medical conditions associated with the individual may comprise one or more of diabetes, insulin resistance, hypoglycemia, insulinoma, Cushing's syndrome, acromegaly, hypothyroidism, hypertension, dyslipidemia, hyperuricemia, or endothelial dysfunction. As an example, the one or more medical conditions may be determined based on one or more physiological attributes associated with the individual. For example, the one or more physiological attributes may comprise a concentration of insulin in one or more of a vascular volume, an interstitial volume, a hepatic volume, or a renal volume. The one or more physiological attributes associated with the individual may be determined based on the one or more physiological parameters. As an example, the one or more medical conditions may be determined based on an appearance and elimination rate of the hormone in the individual. For example, the appearance and elimination rate of the hormone in the individual may be determined based on the one or more physiological parameters.

In an example, a treatment of the one or more medical conditions may be administered to the individual. For example, the computing device (e.g., device 101, electronic device 102, server 106, or any combination thereof) may cause the treatment of the one or more medical conditions to be administered to the individual based on the one or more physiological parameters. As an example, the treatment may be administered based on the one or more physiological attributes associated with the individual. As an example, the treatment may be administered based on the appearance and elimination rate of the hormone in the individual. The treatment may comprise administering the hormone, or hormone replacement therapy. In an example, the hormone may be administered to the individual when the one or more medical conditions associated with the individual are determined. As an example, the administration of the hormone may be adjusted based on the one or more physiological parameters. For example, an inflow of insulin to the individual may be reduced or increased based on the one or more physiological parameters.

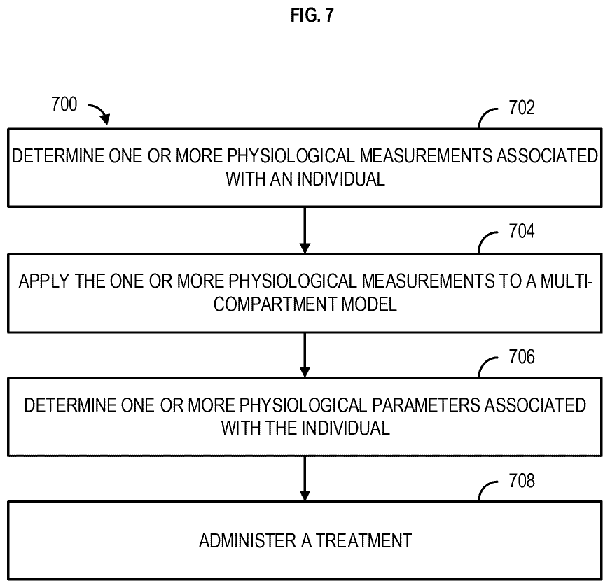

FIG. 7 shows an example method 700 for determining one or more physiological parameters associated with an individual based on one or more physiological measurements associated with the individual. Method 700 may be implemented by computing device (e.g., device 101, electronic device 102, server 106, or any combination thereof). At step 702, one or more physiological measurements associated with an individual may be determined. For example, a computing device (e.g., device 101, electronic device 102, server 106, or any combination thereof) may determine the one or more physiological measurements associated with the individual. The one or more physiological measurements may comprise one or more of plasma insulin concentrations, a plasma volume, a vascular volume, an interstitial volume, a hepatic volume, or a renal volume.

At step 704, the one or more physiological measurements may be applied to a multi-compartment model. For example, the computing device (e.g., device 101, electronic device 102, server 106, or any combination thereof) may apply the one or more physiological measurements to the multi-compartment model. The multi-compartment model may represent a bidirectional flux by diffusion of a hormone between a vascular compartment, an interstitial compartment, a hepatic compartment, and a renal compartment of the individual. The hormone may comprise one or more of insulin, C-peptide, glucagon, amylin, somatostatin, insulin-like growth factors (IGFs), or incretins. The multi-compartment model may comprise four non-linear differential equations. The four non-linear differential equations may represent time-varying concentrations of insulin in a vascular plasma volume, in an interstitial volume, in a hepatic volume, and in a renal volume.

At step 706, one or more physiological parameters associated with the individual may be determined based on the application of the one or more physiological measurements to the multi-compartment model. For example, the computing device (e.g., device 101, electronic device 102, server 106, or any combination thereof) may determine the one or more physiological parameters associated with the individual based on the application of the one or more physiological measurements to the multi-compartment model. For example, the multi-compartment model may be solved, based on the one or more physiological measurements, for the one or more physiological parameters. The one or more physiological parameters may comprise one or more of an interstitial insulin elimination rate (αᵢ), a hepatic insulin elimination rate (αₕ), a renal insulin elimination rate (αₖ), or a permeability constant related to insulin diffusion (k).

At step 708, a treatment may be administered to the individual based on the one or more physiological parameters. For example, the computing device (e.g., device 101, electronic device 102, server 106, or any combination thereof) may cause the treatment to be administered to the individual based on the one or more physiological parameters. The treatment may comprise administering the hormone, or hormone replacement therapy. In an example, the hormone may be administered to the individual when the one or more medical conditions associated with the individual are determined. As an example, the administration of the hormone may be adjusted based on the one or more physiological parameters. For example, an inflow of insulin to the individual may be reduced or increased based on the one or more physiological parameters.

In an example, one or more medical conditions may be determined based on the one or more physiological parameters. For example, the computing device (e.g., device 101, electronic device 102, server 106, or any combination thereof) may determine the one or more medical conditions based on the one or more physiological parameters. The one or more medical conditions associated with the individual may comprise one or more of diabetes, insulin resistance, hypoglycemia, insulinoma, Cushing's syndrome, acromegaly, hypothyroidism, hypertension, dyslipidemia, hyperuricemia, or endothelial dysfunction. As an example, the one or more medical conditions may be determined based on one or more physiological attributes associated with the individual. For example, the one or more physiological attributes may comprise a concentration of insulin in one or more of a vascular volume, an interstitial volume, a hepatic volume, or a renal volume. The treatment may be administered based on the one or more physiological attributes associated with the individual. As an example, the treatment may be administered based on an appearance and elimination rate of the hormone in the individual.

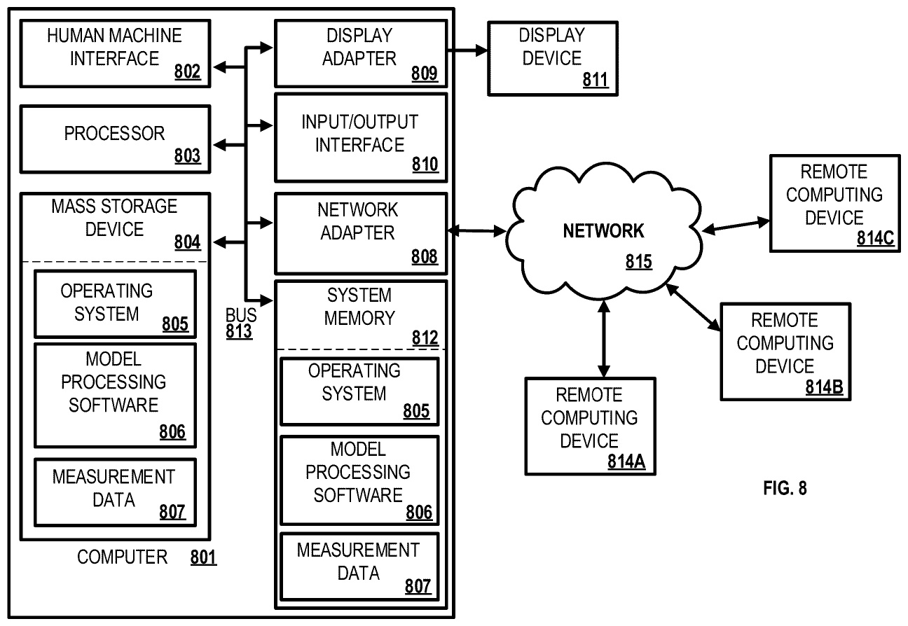

FIG. 8 a block diagram of another example computing device. In an exemplary aspect, the methods and systems can be implemented on a computer 801 as illustrated in FIG. 8 and described below. By way of example, the device 101, the electronic device 102, and the server 106 of FIG. 1 can be a computer 801 as illustrated in FIG. 8. Similarly, the methods and systems disclosed can utilize one or more computers to perform one or more functions in one or more locations. FIG. 8 is a block diagram illustrating an exemplary operating environment 800 for performing the disclosed methods. This exemplary operating environment 800 is only an example of an operating environment and is not intended to suggest any limitation as to the scope of use or functionality of operating environment architecture. Neither should the operating environment 800 be interpreted as having any dependency or requirement relating to any one or combination of components illustrated in the exemplary operating environment 800.

The present methods and systems can be operational with numerous other general purpose or special purpose computing system environments or configurations. Examples of well-known computing systems, environments, and/or configurations that can be suitable for use with the systems and methods comprise, but are not limited to, personal computers, server computers, laptop devices, and multiprocessor systems. Additional examples comprise set top boxes, programmable consumer electronics, network PCs, minicomputers, mainframe computers, distributed computing environments that comprise any of the above systems or devices, and the like.

The processing of the disclosed methods and systems can be performed by software components. The disclosed systems and methods can be described in the general context of computer-executable instructions, such as program modules, being executed by one or more computers or other devices. Generally, program modules comprise computer code, routines, programs, objects, components, data structures, and/or the like that perform particular tasks or implement particular abstract data types. The disclosed methods can also be practiced in grid-based and distributed computing environments where tasks are performed by remote processing devices that are linked through a communications network. In a distributed computing environment, program modules can be located in local and/or remote computer storage media including memory storage devices.

Further, one skilled in the art will appreciate that the systems and methods disclosed herein can be implemented via a general-purpose computing device in the form of a computer 801. The computer 801 can comprise one or more components, such as one or more processors 803, a system memory 812, and a bus 813 that couples various components of the computer 801 including the one or more processors 803 to the system memory 812. In the case of multiple processors 803, the system can utilize parallel computing.

The bus 813 can comprise one or more of several possible types of bus structures, such as a memory bus, memory controller, a peripheral bus, an accelerated graphics port, and a processor or local bus using any of a variety of bus architectures. By way of example, such architectures can comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects (PCI), a PCI-Express bus, a Personal Computer Memory Card Industry Association (PCMCIA), Universal Serial Bus (USB) and the like. The bus 813, and all buses specified in this description can also be implemented over a wired or wireless network connection and one or more of the components of the computer 801, such as the one or more processors 803, a mass storage device 804, an operating system 805, model processing software 806, measurement data 807, a network adapter 808, system memory 812, an Input/Output Interface 810, a display adapter 809, a display device 811, and a human machine interface 802, can be contained within one or more remote computing devices 814A-814C at physically separate locations, connected through buses of this form, in effect implementing a fully distributed system.

The computer 801 typically comprises a variety of computer readable media. Exemplary readable media can be any available media that is accessible by the computer 801 and comprises, for example and not meant to be limiting, both volatile and non-volatile media, removable and non-removable media. The system memory 812 can comprise computer readable media in the form of volatile memory, such as random access memory (RAM), and/or non-volatile memory, such as read only memory (ROM). The system memory 812 typically can comprise data such as measurement data 807 and/or program modules such as operating system 805 and interference processing software 806 that are accessible to and/or are operated on by the one or more processors 803.

In another aspect, the computer 801 can also comprise other removable/non-removable, volatile/non-volatile computer storage media. By way of example, the computer 801 can comprise a mass storage device 804 which can offer non-volatile storage of computer code, computer readable instructions, data structures, program modules, and other data for the computer 801. For example, a mass storage device 804 can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Optionally, any number of program modules can be stored on the mass storage device 804, including by way of example, an operating system 805 and model processing software 806. One or more of the operating system 805 and model processing software 806 (or some combination thereof) can comprise elements of the programming and the model processing software 806. Measurement data 807 can also be stored on the mass storage device 804. Measurement data 807 can be stored in any of one or more databases known in the art. Examples of such databases comprise, DB2®, Microsoft® Access, Microsoft® SQL Server, Oracle®, mySQL, PostgreSQL, and the like. The databases can be centralized or distributed across multiple locations within the network 815.

In another aspect, the user can enter commands and information into the computer 801 via an input device (not shown). Examples of such input devices comprise, but are not limited to, a keyboard, pointing device (e.g., a computer mouse, remote control), a microphone, a joystick, a scanner, tactile input devices such as gloves, and other body coverings, motion sensor, and the like These and other input devices can be connected to the one or more processors 803 via a human machine interface 802 that is coupled to the bus 813, but can be connected by other interface and bus structures, such as a parallel port, game port, an IEEE 1394 Port (also known as a Firewire port), a serial port, network adapter 808, and/or a universal serial bus (USB).

In yet another aspect, a display device 811 can also be connected to the bus 813 via an interface, such as a display adapter 809. It is contemplated that the computer 801 can have more than one display adapter 809 and the computer 801 can have more than one display device 811. For example, a display device 811 can be a monitor, an LCD (Liquid Crystal Display), light emitting diode (LED) display, television, smart lens, smart glass, and/or a projector. In addition to the display device 811, other output peripheral devices can comprise components such as speakers (not shown) and a printer (not shown) which can be connected to the computer 801 via Input/Output Interface 810. Any step and/or result of the methods can be output in any form to an output device. Such output can be any form of visual representation, including, but not limited to, textual, graphical, animation, audio, tactile, and the like. The display 811 and computer 801 can be part of one device, or separate devices.

The computer 801 can operate in a networked environment using logical connections to one or more remote computing devices 814A, 814B, and 814C. By way of example, a remote computing device 814A-814C can be a personal computer, a computing station (e.g., a workstation), a portable computer (e.g., a laptop, a mobile phone, a tablet device), a smart device (e.g., a smartphone, a smart watch, an activity tracker, a smart apparel, a smart accessory), a security and/or monitoring device, a server, a router, a network computer, a peer device, an edge device or other common network node, and so on. Logical connections between the computer 801 and a remote computing device 814A-814C can be made via a network 815, such as a local area network (LAN) and/or a general wide area network (WAN). Such network connections can be through a network adapter 808. A network adapter 808 can be implemented in both wired and wireless environments. Such networking environments are conventional and commonplace in dwellings, offices, enterprise-wide computer networks, intranets, and the Internet.

For purposes of illustration, application programs and other executable program components such as the operating system 805 are illustrated herein as discrete blocks, although it is recognized that such programs and components can reside at various times in different storage components of the computer 801, and are executed by the one or more processors 803 of the computer 801. An implementation of model processing software 806 can be stored on or transmitted across some form of computer readable media. Any of the disclosed methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” can comprise volatile and non-volatile, removable and non-removable media implemented in any methods or technology for storage of information such as computer readable instructions, data structures, program modules, or other data. Exemplary computer storage media can comprise RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

The methods and systems can employ artificial intelligence (AI) techniques such as machine learning and iterative learning. Examples of such techniques include, but are not limited to, expert systems, case based reasoning, Bayesian networks, behavior based AI, neural networks, fuzzy systems, evolutionary computation (e.g., a genetic algorithms), swarm intelligence (e.g., an ant algorithms), and hybrid intelligent systems (e.g., expert inference rules generated through a neural network or production rules from statistical learning).

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

1. A method comprising:
determining, by a computing device, one or more physiological measurements associated with an individual;
applying the one or more physiological measurements to a multi-compartment model, wherein the multi-compartment model represents a bidirectional flux by diffusion of a hormone between a vascular compartment, an interstitial compartment, a hepatic compartment, and a renal compartment of the individual;
determining, based on the application of the one or more physiological measurements to the multi-compartment model, one or more physiological parameters associated with the individual; and
determining, based on the one or more physiological parameters, one or more medical conditions associated with the individual.

2. The method of claim 1, wherein the one or more physiological measurements comprise one or more of plasma insulin concentrations, a plasma volume, a vascular volume, an interstitial volume, a hepatic volume, or a renal volume.

3. The method of claim 1, wherein the multi-compartment model comprises four non-linear differential equations, wherein the four non-linear differential equations represent time-varying concentrations of insulin in a vascular plasma volume, in an interstitial volume, in a hepatic volume, and in a renal volume.

4. The method of claim 1, wherein the hormone comprises one or more of insulin, C-peptide, glucagon, amylin, somatostatin, insulin-like growth factors (IGFs), or incretins.

5. The method of claim 1, wherein the one or more physiological parameters comprise one or more of an interstitial insulin elimination rate (αᵢ), a hepatic insulin elimination rate (αₕ), a renal insulin elimination rate (αₖ), or a permeability constant related to insulin diffusion (k).

6. The method of claim 1, wherein determining, based on the one or more physiological parameters, the one or more medical conditions associated with the individual comprises:
determining, based on the one or more physiological parameters, one or more physiological attributes associated with the individual; and
determining, based on the one or more physiological attributes associated with the individual, the one or more medical conditions associated with the individual.

7. The method of claim 6, wherein the one or more physiological attributes comprise a concentration of insulin in one or more of a vascular volume, an interstitial volume, a hepatic volume, or a renal volume.

8. The method of claim 1, wherein determining, based on the one or more physiological parameters, the one or more medical conditions associated with the individual comprises:
determining, based on the one or more physiological parameters, an appearance and elimination rate of the hormone in the individual; and
determining, based on the appearance and elimination rate of the hormone in the individual, the one or more medical conditions associated with the individual.

9. The method of claim 1, wherein the one or more medical conditions associated with the individual comprise one or more of diabetes, insulin resistance, hypoglycemia, insulinoma, Cushing's syndrome, acromegaly, hypothyroidism, hypertension, dyslipidemia, hyperuricemia, or endothelial dysfunction.

10. The method of claim 1, further comprising causing a treatment of the one or more medical conditions associated with the individual, wherein the treatment comprises administering the hormone, or hormone replacement therapy.

11. A method comprising:
determining, by a computing device, one or more physiological measurements associated with an individual;
applying the one or more physiological measurements to a multi-compartment model, wherein the multi-compartment model represents a bidirectional flux by diffusion of a hormone between a vascular compartment, an interstitial compartment, a hepatic compartment, and a renal compartment of the individual;
determining, based on the application of the one or more physiological measurements to the multi-compartment model, one or more physiological parameters associated with the individual; and
administering, based on the one or more physiological parameters, a treatment associated with one or more medical conditions associated with the individual.

12. The method of claim 11, wherein the one or more physiological measurements comprise one or more of plasma insulin concentrations, a plasma volume, a vascular volume, an interstitial volume, a hepatic volume, or a renal volume.

13. The method of claim 11, wherein the multi-compartment model comprises four non-linear differential equations, wherein the four non-linear differential equations represent time-varying concentrations of insulin in a vascular plasma volume, in an interstitial volume, in a hepatic volume, and in a renal volume.

14. The method of claim 11, wherein the hormone comprises one or more of insulin, C-peptide, glucagon, amylin, somatostatin, insulin-like growth factors (IGFs), or incretins.

15. The method of claim 11, wherein the one or more physiological parameters comprise one or more of an interstitial insulin elimination rate (αᵢ), a hepatic insulin elimination rate (αₕ), a renal insulin elimination rate (αₖ), or a permeability constant related to insulin diffusion (k).

16. The method of claim 11, wherein determining, based on the one or more physiological parameters, the one or more medical conditions associated with the individual comprises:
determining, based on the one or more physiological parameters, one or more physiological attributes associated with the individual; and
administering, based on the one or more physiological attributes associated with the individual, the treatment associated with the one or more medical conditions associated with the individual.

17. The method of claim 16, wherein the one or more physiological attributes comprise a concentration of insulin in one or more of a vascular volume, an interstitial volume, a hepatic volume, or a renal volume.

18. The method of claim 11, wherein determining, based on the one or more physiological parameters, the one or more medical conditions associated with the individual comprises:
determining, based on the one or more physiological parameters, an appearance and elimination rate of the hormone in the individual; and
administering, based on the appearance and elimination rate of the hormone in the individual, the treatment associated with the one or more medical conditions associated with the individual.

19. The method of claim 11, wherein the one or more medical conditions associated with the individual comprise one or more of diabetes, insulin resistance, hypoglycemia, insulinoma, Cushing's syndrome, acromegaly, hypothyroidism, hypertension, dyslipidemia, hyperuricemia, or endothelial dysfunction.

20. The method of claim 11, wherein the treatment comprises administering the hormone, or hormone replacement therapy.
